(19) United States
(12) Patent Application Publication
(10) Pub. No.: US 2002/0045243 A1
(43) Pub. Date: Apr. 18, 2002

(54) Fluid cartridge and method

(76) Inventors: Ronald C. Laska, Minnetonka, MN (US); Vince Fischer, Lake Elmo, MN (US); Greg Thune, Westerville, OH (US); Dwight Musgrave, Granville, OH (US); Kenneth Uffenheimer, Los Gatos, CA (US); Jeffrey Brown, North Logan, UT (US)

Correspondence Address: MERCHANT & GOULD PC, P.O. BOX 2903, MINNEAPOLIS, MN, 55402-0903, US

(21) Appl. No.: 09/952,115

(22) Filed: Sep. 10, 2001

Related U.S. Application Data
(60) Provisional application No. 60/287,553, filed on Sep. 15, 2000.

(51) Int. Cl. C12M 1/34; B01D 15/08

(52) U.S. Cl. 435/287.1; 210/656

(57) ABSTRACT

Fluid flow cartridges, systems and methods for qualitative or quantitative analysis of a sample material are disclosed. Cartridges of the invention are constructed for fluid flow into, out of and internally throughout the cartridges. The cartridges can be used with an analytical instrument configured to interface with the instrument and present the sample or a reagent fluid at a location and in a form appropriate for analysis of the sample by the instrument. The invention is particularly advantageous for use in analytical systems in many fields but may also be used for non-analytical purposes.

### FIELD OF THE INVENTION

The invention is directed to analyte detection or quantification systems. The invention includes cartridges, valve systems, methods and instrumentation systems providing qualitative or quantitative analysis of an analyte in a sample.

### BACKGROUND OF THE INVENTION

Various types of collection, treatment or detection procedures are used for qualitative or quantitative analysis of body fluids, cell cultures, environmental samples, food products, manufacturing process fluids, etc. Clinical chemistry is one example of a discipline that routinely involves collecting, processing and analyzing a sample for detection or characterization of an analyte in body fluids such as blood, urine, spinal fluid, synovial fluid, etc. Typically, analysis is performed by automated instruments utilizing optical or non-optical analyte detection systems.

Common to most analytical systems is the need for simple, safe and efficient sample collection and transfer to the analytical instrument without sample contamination or exposure of laboratory personnel to infectious or toxic materials. Further, proper sample handling directly affects the accuracy of the results obtained. This is particularly important in the health care field where erroneous results can be detrimental to the proper diagnosis, treatment or monitoring of a pathological condition. Accuracy is equally important in industries such as pharmaceuticals, food processing, environmental testing, etc., where analytical results provide the basis for ensuring product quality, consistency or wholesomeness.

In further advancement of clinical diagnostics and other fields, the present invention provides simple, safe and efficient sample handling and processing procedures for accurate, reliable and repeatable results.

### SUMMARY OF THE INVENTION

The invention provides cartridges, analytical instrument systems and methods for simple, safe and efficient handling and analysis of a sample. The cartridges of the invention can be disposable or reusable and are typically constructed for fluid flow into, out of, and internally throughout. The cartridges can be manually operated or used with an analytical instrument.

In one embodiment, the invention provides a cartridge for sample analysis. The cartridge can include one or more fluid reservoirs for storing a fluid on the cartridge, a plurality of fluid passageways, a selector valve for selectively controlling fluid flow through the fluid passageways and an analyzer interface region for interfacing with a detector. The cartridge can also comprise a sample receiving region for receiving a sample of material. The sample receiving region can be removable or integral to the cartridge. The cartridge can also include a waste receptacle which is removable or integral to the cartridge. The cartridge can also include a conditioning region for interfacing with a fluid conditioning element to condition a fluid passing through the passageways.

A selector valve of the cartridge provides for selectively controlling fluid flow through the fluid passageways of the cartridge. The selector valve can be automatically controlled by an analytical instrument or manually controlled by an operator.

The invention also provides sample analysis systems including analytical instruments operating with a cartridge of the invention. It will be appreciated that the principals and methods of the invention can be used with various different types of diagnostic and nondiagnostic instrumentation.

### BRIEF DESCRIPTION OF THE DRAWINGS

FIG. 1 is a schematic diagram of fluid flow passages of a sample cartridge according to the invention;

FIG. 2 is a top perspective view of one embodiment of a cartridge according to the invention;

FIG. 3 is a top plan view of the cartridge of FIG. 2;

FIG. 4 is a bottom perspective view of the cartridge of FIG. 2;

FIG. 5 is a bottom plan view of the cartridge of FIG. 2;

FIG. 6 is a top exploded perspective view of the cartridge of FIG. 2;

FIG. 7 is a bottom perspective view of the body of the cartridge of FIG. 2;

FIG. 8 is a top plan view of the body of the cartridge of FIG. 2;

FIG. 9 is a bottom plan view of the body of the cartridge of FIG. 2;

FIG. 10 is an enlarged top plan view of the valve region of the body of the cartridge of FIG. 2;

FIG. 11 is a perspective view of one embodiment of a valve core operable with the valve seal of FIG. 12;

FIG. 12 is a perspective view of a valve seal according to one embodiment of the invention;

FIG. 13 is a cross-section view of a body of the invention taken through line 13-13 of FIG. 8;

5 FIG. 14 is an enlarged view of a portion of the cross-section view of the body of FIG. 13;

FIG. 15 is a top perspective view of an embodiment of a docking arrangement of the invention;

FIG. 16 is a top plan view of the docking arrangement of FIG. 15;

FIG. 17 is a first side plan view of the docking arrangement of FIG. 15; and

FIG. 18 is a bottom plan view of the docking arrangment of FIG. 15.

### DETAILED DESCRIPTION

The invention provides cartridges, systems and methods for qualitative or quantitative analysis of a sample material including determining the presence, absence, quantity, concentration, characteristics, color, viscosity, etc., of an analyte in the sample. Cartridges of the invention can be disposable or reusable and are typically constructed for fluid flow into, out of, and internally throughout the cartridge. The cartridges can be manually operated or used with an analytical instrument and configured to interface with the instrument and present the sample at a location and in a form appropriate for analysis of the sample by the instrument. The invention is applicable for use in analytic systems in many fields as well as for fluid delivery and dispensing purposes outside analytic uses.

It will be noted that in several places throughout the specification, guidance is provided through lists of examples. In each instance, the recited list serves only as a representative group. It is not meant, however, that the list is exclusive.

As used herein and the appended claims, the singular forms of the articles “a,” “an” and “the” include plural referents unless the content clearly dictates otherwise. Thus, for example, reference to “a reagent” includes a single or multiple reagents, reference to “a fluid reservoir” includes reference to one, two or more of such reservoirs, etc.

The cartridges of the invention can be constructed and configured for manual use or for use with a particular analytical instrument and nothing in this disclosure should be construed as limiting the invention to only those types of instruments expressly discussed herein. The cartridges can be manufactured using any known method including pour molding, injection molding, machining, etc. In the illustrated embodiments, the fluid passageways through the cartridges include surface passageways, through body passageways and surface vias advantageously configured for manufacture using known injection molding techniques. As will be described, fluid is maintained within surface channels to form surface passageways by use of surface covers including membranes manufactured from plastics, foils, etc., which may be of varied thickness or rigidity,

Analytical instruments and instrument systems suitable for use according to the invention provide sample analysis utilizing various technologies including, for example, optical detection systems, such as colorimetry, ultraviolet and visible spectrophotometry, infrared spectrometry, Raman spectrometry, internal and external reflection methods, such as, ellipsometry, external Brewster angle reflectometry, evanescent wave reflectometry, critical-angle reflectometry, evanescent wave ellipsometry, surface plasmon resonance, scattered total internal reflection, optical waveguide sensing methods, refractometric optical fiber sensing methods, leaky waveguide sensing methods, resonance light scattering of particles, multilayered grating resonance, diffraction anomaly grating methods, etc. In some assays, optical detection may also be performed by the human eye. Technologies not relying on optical detection, such as ion selective electrode devices, flow-restriction measurement, capacitance, etc., can also be used.

As used herein, the “detector” of an analytical instrument is that portion of the instrument to which a sample (or a product of the sample, such as a reaction product of the sample and a reagent) is presented for actual determination of the presence, absence, quantity, character or other parameter of the analyte for which the sample is being analyzed. It should be understood that although the invention discusses analysis of a sample and presentation of the sample to a detector, the detector could alternatively be a “treatment arrangement” that acts on a sample material or other fluid material for treatment, reaction, purification, etc., of the material presented to the detector. Also, as stated above, the detector can be a human observing or performing some other activity on the material presented.

The “control center” of an analytical instrument can be manually operated or, typically, a software driven operating system that controls the operation of the cartridge within the instrument including control of timing, sequence, direction, volume, heating, cooling or other parameter related to flow, conditioning or selection of a fluid passing through the cartridge. The control center can be internal or peripheral to the instrument. Indeed, it will be appreciated that many features of an analytical instrument which are herein described for interfacing with the cartridge need not be integral to the instrument but may be performed by peripheral equipment that operates in conjunction with the instrument. Thus, discussion of features of an analytical instrument as a component of the instrument should not be construed as limiting the invention but rather as one means for embodying the interactive features of the invention.

For exemplary purposes herein, the invention may be described with reference to surface plasmon resonance (“SPR”) systems. However, it will be appreciated that the invention is not limited to SPR systems. Suitable SPR systems are known and described in, for example, in U.S. Pat. Nos. 4,931,384; 4,828,387; 4,882,288; 4,992,385; 5,118,608; 5,164,589; 5,310,686; 5,313,264; 5,341,215; 5,492,840; 5,641,640; 5,716,854; 5,753,518; 5,898,503; 5,912,456; 5,926,284; 5,944,150; 5,965,456; 5,972,612; and 5,986,762, PCT Patent Applications Publication Nos. WO 88/07202 and WO 88/10418, and UK Patent Application Publication No. GB 2 202 045, all of which are incorporated herein by reference.

As used herein, when referring to the “analysis” of a sample, sample material or analyte, the term “analysis” means the detection, quantification, characterization, amplification, treatment, or other procedure that may be performed on a sample or analyte manually, or by the particular instrument with which the invention is used. An “analyte” includes any component that is or may be present in a sample for which an “analysis” may be performed including, for example, proteins, peptides, ions, electrolytes, molecules, hormones, enzymes, carbohydrates, toxins, receptors, etc. Other analyses for which a cartridge may be used in clinical chemistry include, for example, CBC, coagulation, etc.

A “sample” or “sample material” includes any material for which an analysis may be performed. Typically, the sample will be in a fluid (i.e., in a flowable form). If the sample is in a dry form it can be put into a more readily flowable form using a suitable carrier to form a solution, suspension, emulsion, etc., before or after the sample is introduced into the cartridge. According to the invention, a sample includes, for example, any physiological or pathological body fluid from a human or animal including serum, plasma, urine secretions, excretions, exudates, transudates, cell suspensions including blood, lymph, synovial fluid, spinal fluid, semen, saliva containing buccal cells, skin scrapings, hair root cells, etc. A “sample” also includes any physiological or pathological fluids or cell suspensions from plants; extracts or suspensions of bacteria, fungi, plasmids, viruses etc.; products, extracts or suspensions of parasites including helminths, protozoas, spirochetes, etc.; liquid extracts or homogenates of human, animal or plant tissues (e.g., bone, liver, kidney, etc.); media from DNA or RNA synthesis, amplification or media from antibody production; environmental, agricultural, or food processing materials; water samples, fuels, beverages, gaseous materials; etc.

In some embodiments, the invention is particularly suited for use in the field of health care, for example, clinical chemistry. Thus, for exemplary purposes, the invention can be described with respect to the field of clinical chemistry and clinical chemistry instrumentation but in no way is it intended that the invention be limited solely to this use.

In one embodiment, the invention provides for introducing a sample into a cartridge, inserting the cartridge into an analytical instrument, analyzing the sample and removing the cartridge from the instrument after analysis without having to directly contact the sample or sample container after collection. In addition, because all samples can be processed entirely within the cartridge, no sample, reagent or other component is introduced into the instrument thus reducing the possibility of instrument or cross sample contamination. Such embodiments may be particularly advantageous for doctors offices or emergency room use.

In most embodiments a cartridge will contain all reagents or other components necessary to perform a single or multiple analyses. Thus, various cartridges containing the reagents or other components necessary to perform a particular analysis, will be available. Alternatively, the cartridge may contain some or none of the necessary reagents for an analysis. In this case, the sample or fluid necessary for a particular analysis can be introduced into the cartridge by the instrument or a human as needed.

Throughout the disclosure, reference will be made to “fluid passageways”. The passageways provide for fluid movement into, out of, or throughout the cartridge whether the fluid is a sample material, reagent, “driver” or other fluid. As used herein, a “driver” moves fluids, such as a reagent or a sample material through the passageways. The driver is a flowable material and generally includes inert fluids. As used herein, an “inert fluid” includes liquids, gases or other flowable material that does not alter a sample, reagent or analyte in such a way as to cause error in the analysis if the driver contacts one or more of them. Examples of typical drivers include air, nitrogen, helium, argon, etc. For most purposes, air may be a suitable driver and thus will be the exemplary driver used for discussion herein.

A “reagent” has its commonly known meaning and includes fluids used in processing, reacting, washing, modifying, etc., a sample, analyte or the cartridge for a particular assay.

In a clinical chemistry laboratory setting, the sample collection container, such as a blood collection tube, can be mounted to the cartridge and the sample introduced into the cartridge directly from the sample collection container without contact by laboratory personnel. In alternative embodiments, the sample can be transferred from the collection container to an integral sample reservoir on the cartridge prior to insertion of the cartridge into the instrument. After completion of the analysis, the spent cartridge and sample container can be disposed of pursuant to governing disposal rules and regulations. In the event that it is desired to remove the collection container from the cartridge prior to disposal, such as for archival purposes, in another embodiment the invention provides convenient docking arrangements for mounting or detaching the collection container from the cartridge with minimal contact of the collection container.

The sample can be passed through the cartridge along fluid passageways which direct the sample to an analyzer interface region for detection of the analyte by the detector of the instrument. Reagents or other non-sample components necessary for a particular analysis are contained in reservoirs on the cartridge and are also passed through fluid passageways to the analyzer interface region. Some fluid passageways are commonly traveled by all fluids of the cartridge and others may be used exclusively by the sample material or a particular reagent from a particular reservoir. “Mixing reservoirs” can be present to mix individual or multiple reagents or mix reagents with the sample prior to directing the sample material or reagent to the analyzer interface region.

The cartridge can also include a waste receptacle to collect used reagents, excess sample material or sample components washed from the sample by the reagents. If present, the waste receptacle can be integral to the cartridge or a separable component. In preferred embodiments, a waste receptacle is an integral part of the cartridge.

In a typical embodiment, the driver enters the cartridge at a time, volume and rate controlled by the control center for a particular assay to move the fluid (e.g., sample material or reagent) through the cartridge by volumetric displacement. The driver enters the cartridge through a driver interface present between the instrument and the cartridge. The driver can be provided by the instrument and introduced into the cartridge through a driver port by a pump such as a mechanical pump, electrical pump, compressor, single stroke positive displacement pump (e.g., a syringe, thumb pump), etc. The driver can alternatively be provided by a pump on the cartridge operated by the instrument (e.g., disposable septum) or external to the cartridge and operated by a human.

The cartridge can also include a selector valve. As used herein, a “selector valve” is a valve that can be selectively positioned to direct fluid: entering the valve from a single inflow passage, back out a selected one of a plurality of outflow passages; to a single outflow passage from a selected one of a plurality of inflow passages; or both. In a preferred embodiment, a selector valve can receive the fluid from a selected one of a plurality of inflow channels and provide exit through a selected one of a plurality of outflow passages.

In a typical embodiment, once through the driver port, the driver, e.g., air, moves along a driver inflow passageway into the valve chamber in the valve region. Once in the valve chamber, the valve position directs the air to a selected sample or reagent reservoir inflow passageway. At least one dedicated inflow passageway and one dedicated outflow passageway is present for the sample reservoir, regardless of whether the sample reservoir is separable or integral to the cartridge, and for each reagent reservoir. Positioning of the valve to direct the air to the sample reservoir or to a particular reagent reservoir is controlled by the control center of the instrument and may be unique for a particular analysis of a particular cartridge. Depending on the volume of air, the air drives some or all of the fluid from the selected reservoir into the dedicated reservoir outflow passageway for that reservoir and back to the valve region. At the valve region, the fluid is then directed to the analyzer passageway. The analyzer passageway guides the fluid to the analyzer interface region of the cartridge. The analyzer interface region includes a passageway to direct the fluid to a flow cell, cuvette, analysis or other reading arrangement from which the sample will be detected by the detector. As will be described, in one embodiment, the valve position that directs the air to a particular sample or reagent inflow passageway is the same valve position that directs the fluid coming from the sample or reagent outflow passageway into the analyzer passageway.

Fluid can then be passed from the analyzer interface region to the waste passageway into the waste receptacle. The waste receptacle is preferably vented to release the air from the cartridge. Alternatively, the waste receptacle can have an expandable volume for receiving the driver or fluids passing from the analyzer interface region.

Thus, for a particular analysis to be performed on a sample in a particular cartridge, the control center (or human operator) can selectively position the valve to drive the through the cartridge in a predetermined sequence along the fluid passageways to the analyzer interface region for detection.

A fluid filter can be positioned along portions of the fluid passageways to filter any fluid passing therethrough. In addition, a fluid conditioning region can be provided on the cartridge along one or more passageways to interface the fluid with a fluid conditioning element on the instrument. For example, the instrument could provide a fluid conditioning element to interface with the conditioning region of the cartridge to heat, cool, aerate, expose to magnetic fields, etc., the fluid as the fluid moves to the analyzer interface region.

The cartridge can also include a label, marked with, for example, a two dimensional or three dimensional bar code, that can be automatically read by the instrument to identify the sample source, the analysis to be performed, the lot number of the cartridge, expiration date and any other information determined to be necessary for a particular application. For some instruments, by indicating the analysis to be performed on the label, the control center can automatically control fluid flow rate, fluid flow volume, fluid sequence, fluid direction, reaction temperatures, etc., for a particular analysis. Alternatively (or additionally), the operator can scan patient information into the instrument from a bar code placed on the collection container prior to introducing the sample material into the cartridge. In an alternative embodiment, patient information, cartridge information and test information can be entered using a keyboard or network communication. Patient information from the label can be stored by the instrument and later associated with the analysis results.

It will be appreciated that while the invention does not require any sample or reagents to come into direct contact with the instrument detector, the instrument and cartridge can have multiple interfaces. Examples of interfaces between the instrument and cartridge include an identification interface where the instrument reads a label affixed to a cartridge; a driver interface where the driver enters the cartridge from the instrument; a selector valve interface where the instrument controls the valve position; a conditioning region interface where a fluid conditioning element of the instrument can condition a fluid passing through the cartridge; and an analyzer region interface where the sample is presented to the instrument for analysis.

### DETAILED DESCRIPTION OF ILLUSTRATED EMBODIMENTS

The invention will be further described with reference to the accompanying drawings, wherein like reference numerals identify identical components throughout the several views. The illustrated embodiments and following description are for exemplary purposes to facilitate comprehension of the invention and should not be construed to limit the scope of the invention.

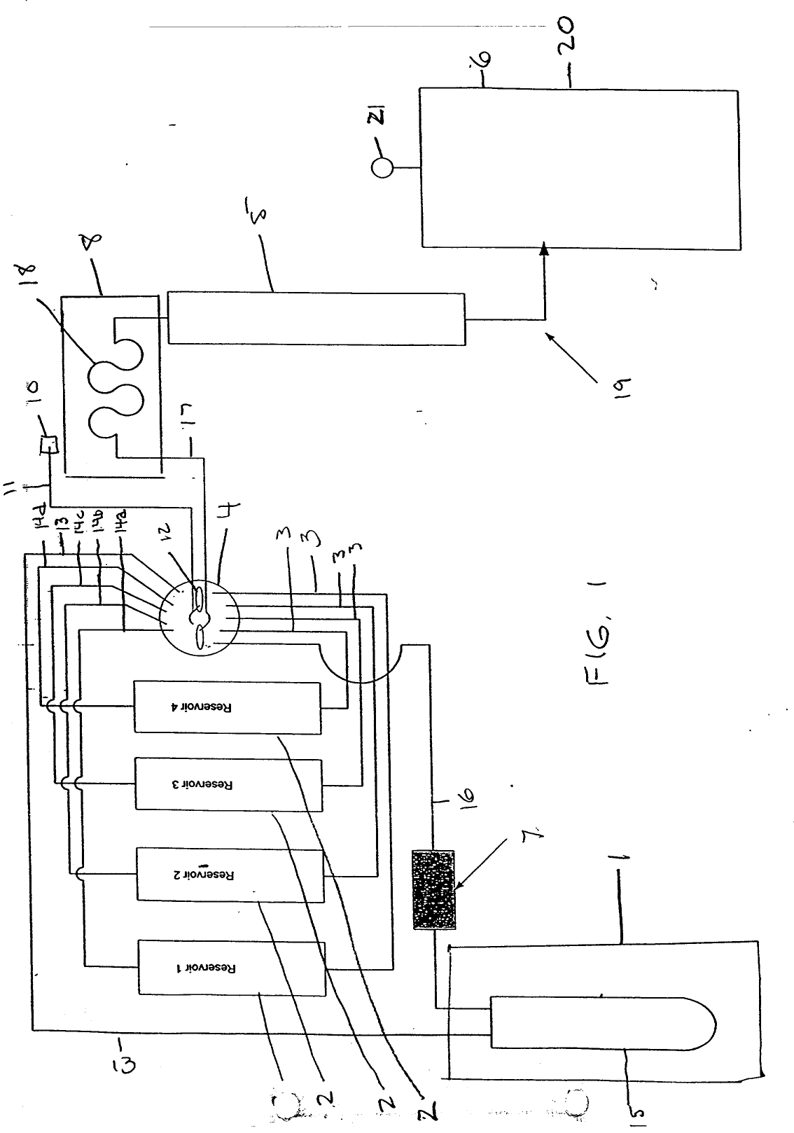

FIG. 1 is a schematic diagram illustrating the various regions, reservoirs, passageways and other features of a sample cartridge according to the invention. As illustrated, a cartridge of the invention includes a sample receiving region 1, one or more reagent reservoirs 2, a plurality of fluid passageways 3, a valve region 4, an analyzer interface region 5 and a waste receptacle 6. In addition, a cartridge can optionally include a fluid filter 7 or a conditioning region 8.

In use, a driver such as air can enter driver input region 10 and move along driver inflow passageway 11 to valve region 4. At valve region 4, valve 12 can be positioned to direct the air into sample inflow passageway 13 or a selected one of reservoir inflow passageways 14a-14d. For exemplary purposes here, assuming valve 12 is positioned to direct air along sample inflow passageway 13, the air moves along sample inflow passageway 13 to sample receiving region 1, in this case a removable collection tube 15, to drive some or all of the sample out of collection tube 15 into sample outflow passageway 16, through fluid filter 7 and back into valve region 4. The position of valve 12 permits the air to then drive the sample material through analyzer passageway 17 into analyzer interface region 5. If a conditioning region 8 is present, analyzer passageway 17 can be configured to have an increased distance of travel, such as undulations 18, to increase the surface area of the fluid exposed to conditioning region 8 as the fluid passes through conditioning region 8. Sample material or other fluid can pass from analyzer region 5 into waste passageway 19 to move the fluid into waste region 6, such as waste receptacle 20. Waste region 6 can include a vent 21 to release the air from the cartridge as the fluid is passed into waste receptacle 20.

By changing the position of valve 12, the air can be selectively directed to move fluid along a similar path from each of the reagent reservoirs 2, along each of the reservoirs' respective inflow and outflow passageways to waste receptacle 19. It will be appreciated that driver inflow passageway 11, valve region 4, analyzer passageway 17 and waste passageway 19 are commonly used for fluid flow from both the sample and reagent reservoirs.

Specific details of various embodiments of a sample cartridge according to the invention will now be further described below.

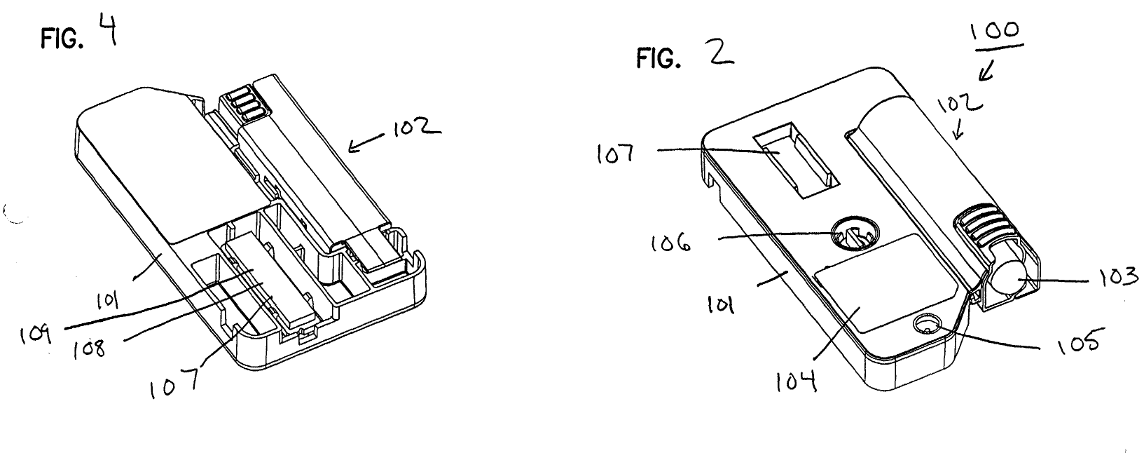

FIG. 2 is a top perspective view of one embodiment of a cartridge 100 according to the invention. As used herein, the terms “top” and “bottom” are not intended to define the orientation of a cartridge 100 when in use. Rather, the terms are for reference purposes for describing the invention. Cartridge 100 includes a body 101, a sample docking arrangement 102 for mounting a sample container 103 to body 101, a label 104, a driver interface region 105, a valve region 106 and a analyzer interface region 107. Body 101 can be prepared from known materials, such as plastics, including polypropylene, high density polyethylene (HDPE), polycarbonate, polyacrylate, etc. The passageways, channels, vias or other openings of the body can be injection molded, pour molded or drilled, e.g., laser drilling, after formation of the body.

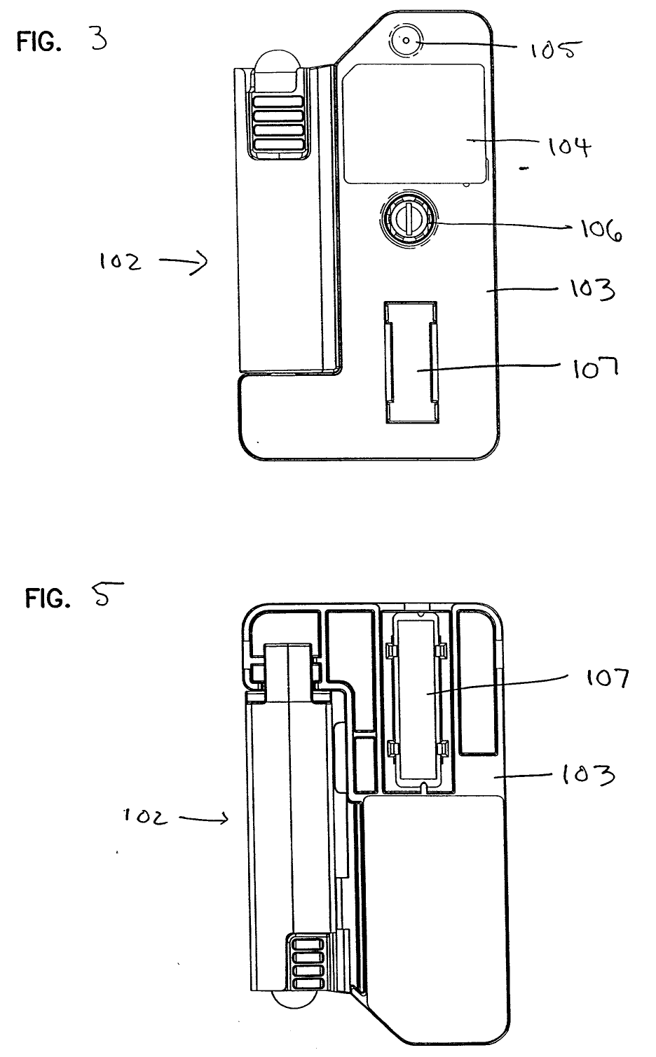

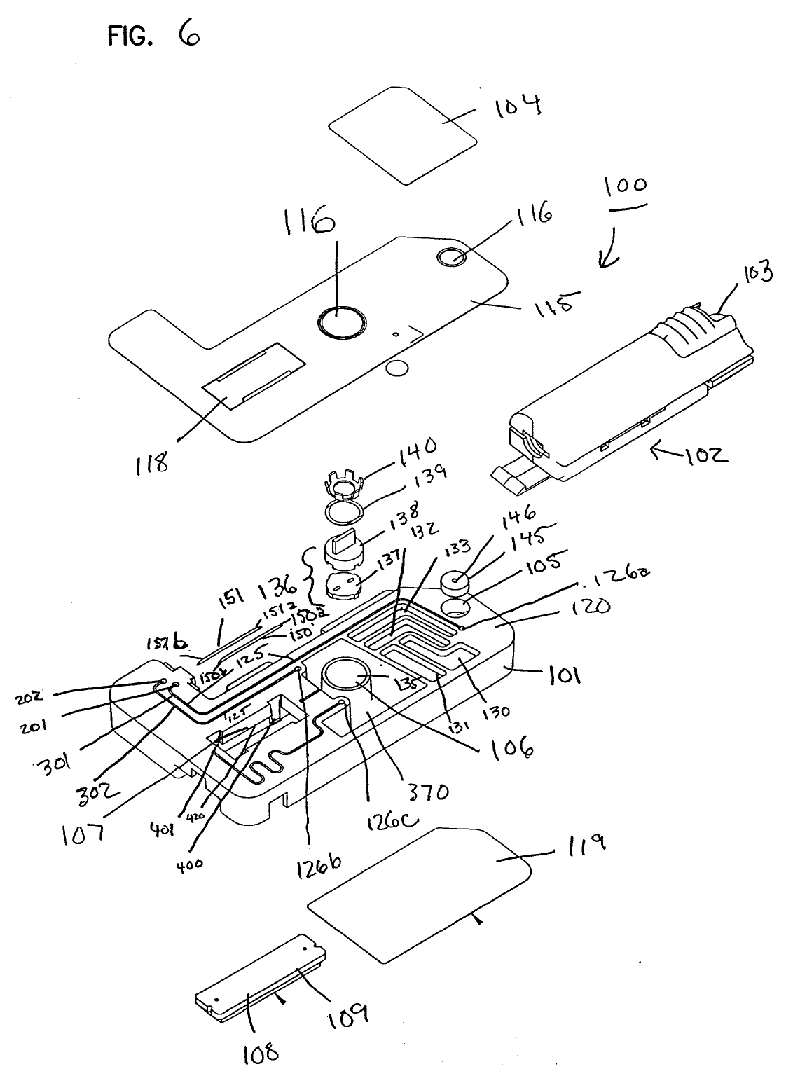

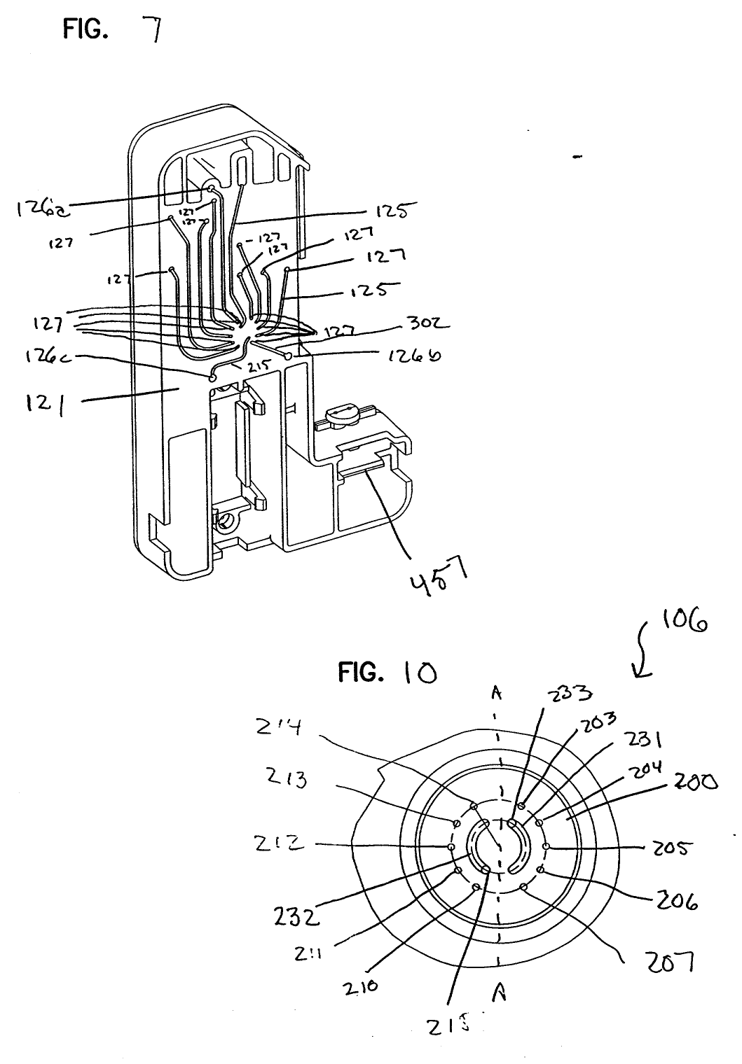

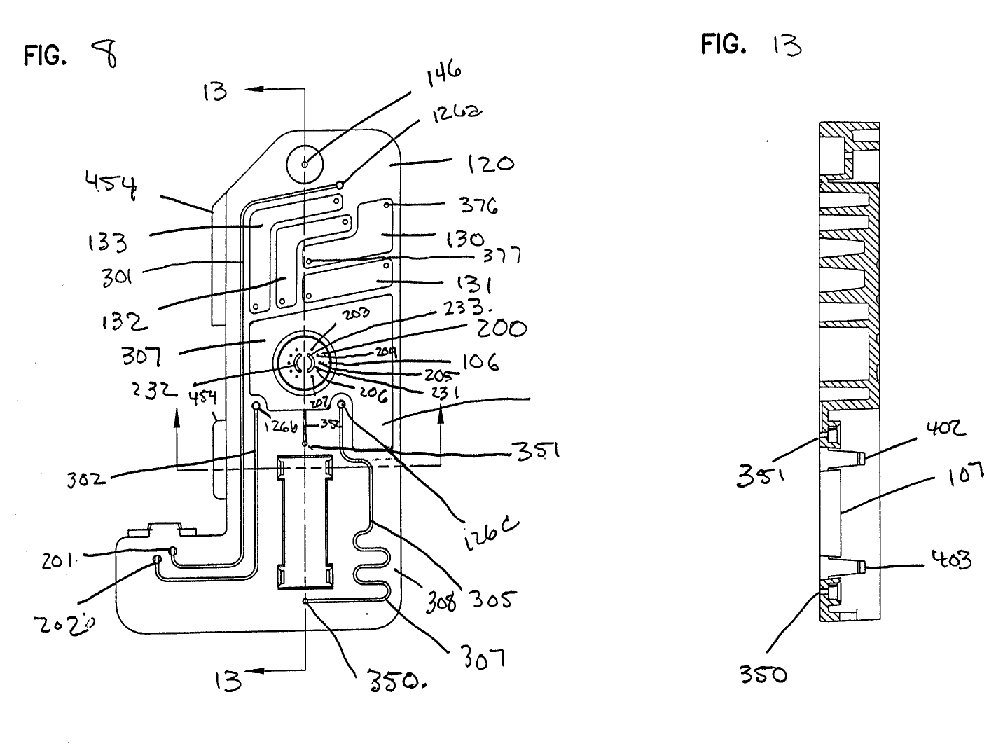

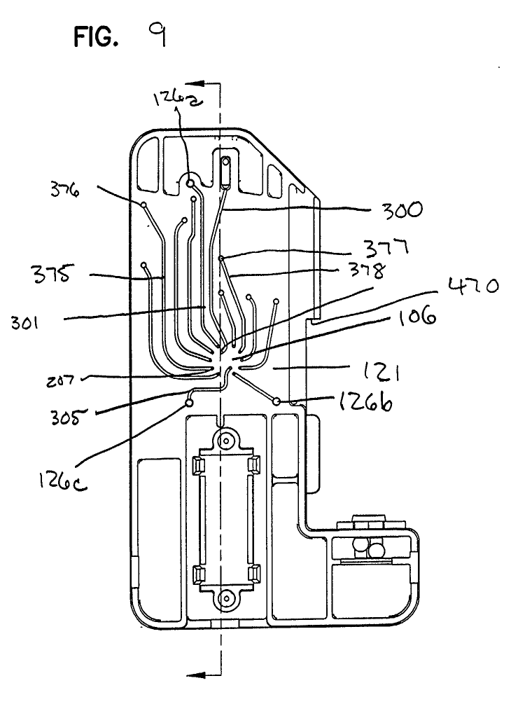

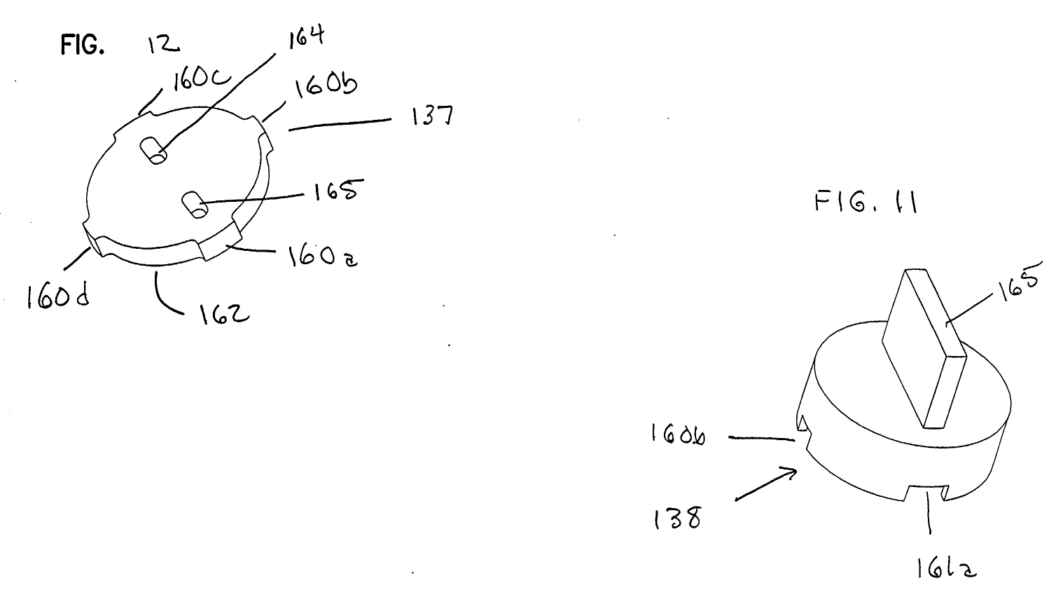

FIG. 3 is a top plan view, FIG. 4 is bottom perspective view, and FIG. 5 is a bottom plan view, of cartridge 100 further illustrating the components described in FIG. 2. In general, FIGS. 1-5, as well as other FIGs. herein, also illustrate the surface and configurations and contours for injection molding of cartridge 100. FIG. 4 also illustrates reading arrangement 108, such as flow cell assembly 109 within analyzer interface region 107. In one embodiment, for use with an SRP instrument, flow cell assembly 109 can include a gold plated transducer having a plastic base (e.g., polycarbonate) and a cover such as Zeonex® available from Zeon, Japan. The reading arrangements 108 can include any chemicals or chemical coatings that may be necessary to perform a particular analysis in a particular cartridge.

FIG. 6 is an exploded view of the cartridge of FIGS. 1-4. As illustrated, label 104 can be mounted to top membrane 115 which is sealably mounted to body 101. Top membrane 115 can be prepared from known transparent or opaque film materials including foils or plastics. As illustrated, top membrane 115 includes a driver interface region opening 116, valve region opening 117 and analyzer interface region opening 118. When top membrane 115 is mounted to top surface 120 of body 101, top membrane 115 provides containment of fluids in the reagent reservoirs 130-133, fluid channels (e.g., passageways 301, 302) and waste receptacle 370. Bottom membrane 119 is sealably mounted to the bottom surface 121 of body 101 and performs a similar function as top membrane 115.

Because cartridge 100 can be a low pressure system (e.g., 0.2 to 2 psig), most known heat sealable membranes, such as polyester, etc., can be used as membranes. Alternatively, known pressure sensitive adhesives can be used.

If cartridge 100 is to be run as a high pressure system, ultrasonic bonding or a molded cover may be advantageous. However, one advantage of a low pressure system is that the volume of air in the cartridge is only slightly compressed and therefore the volume of fluid moved by the air can be more easily controlled.

FIG. 7 is a bottom perspective view of body 101. As illustrated in FIGS. 6 and 7, top surface 120 and bottom surface 121 of body 101 include a plurality of surface channels 125. Sealing top membrane 115 and bottom membrane 116 to their respective body surfaces forms a portion of the fluid passageways (described below) from surface channels 125. Some of surface channels 125 are continuous with through body passageways 126a-126c. Through body passages 126a-126c provide continuous passage from top surface 120 to bottom surface 121. In addition, vias will be discussed which are continuous with surface channels 125 and extend through top or bottom surfaces, such as bottom surface 121 into reagent reservoirs 130-133 and valve region 106.

Four reagent reservoirs, 130-133, are shown in the illustrated embodiments. However, more or less reagent reservoirs can be present as needed for a particular analysis. In the illustrated embodiment, reagent reservoirs 130-133 extend from top surface 120 to bottom surface 121 and are sealed on the bottom by bottom surface 121 and on the top by top member 115.

Valve region 106 includes a valve chamber 135 configured for receiving selector valve 136 comprising valve seal 137 and valve core 138. Selector valve 136 can be maintained in position by seal ring 139 and retaining ring 140.

Driver interface region 105 includes a driver seal 145 including a driver port 146 through which a driver, such as air, can be passed from the instrument (not shown) to the passageways as will be further described below.

Referring to FIG. 6, hollow tubes 150 and 151 extend from body 101 and include first piercing ends 150a and 151a, respectively, for penetrating through a sample container 103, such as a rubber stopper. Proximal ends 150b and 151b are in fluid communication with sample inflow passageway 301 and sample outflow passageway 302, respectively, through sample inflow via 201 and sample outflow via 202, respectively. Passageways providing fluid communication between hollow tube 150 and sample inflow via 201 and between hollow tube 151 and sample inflow via 202 are present but not visible.

FIG. 8 is a top plan view of body 101, FIG. 9 is a bottom plan view of body 101 and FIG. 10 is an enlarged top plan view of valve seat 200 of valve region 106. FIG. 11 is a perspective view of valve core 138 and FIG. 12 is a perspective view of valve seal 137. These and other figures will be used to describe fluid flow through fluid passages of cartridge 100. The previously described fluid channels, through body passageways and vias are all components of the fluid passageways when enclosed by the top membrane 115 and bottom membrane 116. In FIGS. 7-10, the membranes have been removed but fluid flow through the “channels” will nonetheless be referred to as “passageways” for purposes here.

Referring to FIGS. 6, 8, and 10, valve region 106 includes a valve seat 200 having a driver delivery channel 231 and a sample/reagent delivery channel (“SR delivery channel”) 232. Driver delivery channel 231 receives air from driver port 146 through driver inflow via 233. The air is then directed to a selected one of the sample or reservoir inflow passages through valve 136 and into a selected one of driver delivery vias 203-207. Each of vias 203-207 are continuous with a single one of the sample or reservoir inflow passageways. Thus, all vias to the right of line A-A in FIG. 10 only have the driver (e.g., air) passing therethrough. As will be explained below, in a similar manner, sample material or reagent enters SR delivery channel 232 from a sample or reagent outflow channel through SR delivery vias 210-214. Each of SR delivery vias 210-214 are continuous with a single one of the sample or reagent outflow passageways. The sample material or reagent is then directed by valve 136 into analyzer passageway 305 (FIG. 9) through analyzer outflow via 215.

FIGS. 11 and 12 are enlarged perspective views of valve core 138 and valve seal 137, respectively. Valve seal 137 is fixedly mounted to valve core 138 in the orientation shown in FIG. 6. In the illustrated embodiment, valve seal 137 is fixed in position by interdigitation of keys 160a-160d of valve seal 137 with notches 161a-161d (only 161a and 161b visible) of valve core 138. In this embodiment, valve tab 165 is the point of engagement with the instrument and thus tab 165 (and the entire valve core) is preferably prepared from known rigid materials such as polycarbonate, acrylic, etc.

Valve seal 137 includes a sealing surface 162 that is sufficiently compliant to conform to valve seat 200 to provide a seal therewith, but preferably can also slide freely as valve core 138 is rotated within valve chamber 135. Suitable sealing materials are known and include, for example, elastomeric materials such as neoprene, urethane, etc. Preferably, the durometer of the elastomeric material permits conformation to the valve seat without deformation that would permit leaking. In some embodiments, the durometer range is about 30-50 shore A. In one preferred embodiment, the seal has a durometer of about 40 shore A. The sealing surface can preferably include a low friction, high-tensile strength material, such as a 1-2 mil polyester cover, bonded to the sealing surface. Also, in the illustrated embodiment valve 136 comprises a separable seal 137 and core 138. However, valve 136 could alternatively be a unitary piece having a rigid valve and compliant valve seal portion.

Valve seal 137 also includes two radial slots 164 and 165. Thus, when positioned within valve chamber 135 valve slot 164 can provide for fluid communication between driver inflow via 233 and a single one of driver delivery vias 203-207. Likewise, radial slot 165 can provide for fluid communication between analyzer outflow via 215 and a single one of SR delivery vias 210-214. Therefore, when valve 136 is positioned such that slot 164 is aligned over driver inflow via 233 and a single driver delivery via (one of 203-207) and slot 165 is aligned over analyzer outflow via 215 and a single SR delivery via (one of 210-214) flow into or out of all other vias is sealed by valve 136.

With the aid of the above discussion, movement of a sample material from a sample container by a driver will now be described. Under the control of the control center, air enters driver port 146 (FIG. 8) near top surface 120 and passes to driver inflow passageway 300 (FIG. 9) along bottom surface 121 and into driver delivery channel 231 in valve region 106 through driver inflow via 233. Valve 136 is positioned such that radial slot (164 or 165) directs the air back out through driver delivery via 203 into sample inflow passageway 301 along bottom surface 121 (FIG. 9) into through body passageway 126a (FIGS. 7,8) continuing to sample inflow passageway 301 along top surface 120 and then passing into sample inflow via 201 and out hollow tube 150 (FIG. 6). The air then drives sample material out of the sample reservoir (not shown in FIG. 6) into hollow tube 151 and through sample outflow via 202 into sample outflow passageway 302 into through body passageway 126b and along bottom surface 121 (FIG. 9). The sample material then passes through SR delivery via 210 into SR delivery channel 232 and out analyzer via 215 into analyzer passageway 305 on bottom surface 121 into through body passageway 126c to top surface 120 (FIG. 8).

Air then moves through analyzer passageway 305 enters analyzer interface region 107 through analyzer inflow via 350. FIG. 13 is a cross section view taken through line 13-13 of FIG. 8. In FIG. 13, flow cell assembly 109 is not present. However, it will be appreciated that fluid entering analyzer inflow via 350 can pass across the analyzer interface region 107 and exit out analyzer outflow via 351 into waste passageway 352 and finally pass into waste receptacle 370 (FIG. 8).

Note that in the illustrated embodiment, analyzer passageway 305 includes a tortuous path 307 to increase the surface area of the fluid traveling through conditioning region 308. In this embodiment, the conditioning region 308 can be contacted from the bottom side of top surface 120 by a fluid conditioning element (not shown) of the analytical instrument to condition fluid prior to entering the analyzer interface region.

Fluid movement from reagent reservoirs 130-133 travels a similar path as described above for the sample material and will be described using reagent reservoir I 130 as an example. To move a reagent fluid from reagent reservoir 130 to analyzer interface region 107, air is once again introduced into driver port 146 (FIG. 8) and passes to driver inflow passageway 300 (FIG. 9) along bottom surface 121 and into driver delivery channel 231 in valve region 106 through driver inflow via 233. Valve 136 is positioned such that a radial slot (164 or 165) directs the air through driver delivery via 206 into reagent reservoir I inflow passageway 375 along bottom surface 121 (FIG. 9) into reagent reservoir I inflow via 376 (FIGS. 8 and 9) and continuing through reagent reservoir I to push reagent I out of reagent reservoir outflow via 377 into reagent reservoir I outflow passageway 378 into SR delivery via 213. Reagent I is then driven through a radial slot (164 or 165) into SR delivery channel 232 out analyzer via 215, into analyzer passageway 305 and moves along analyzer passageway 305 to the analyzer interface region 107 as described above for the sample material.

Each of the reagents necessary for a particular analysis can be sequentially passed into the analyzer interface region as described for reagent I. After each fluid has passed through the analyzer interface region it is disposed of into waste receptacle 370 as described for the sample. The sequence of the reagents moving to the analyzer interface region is selectively controlled by the position of the valve 136 which is under the control of the control center.

In the illustrated embodiment, each of the reagent reservoir inflow vias and outflow vias (e.g., 376, 377) are positioned along the bottom surface of the reagent reservoirs. However, in alternative embodiments, the reagent inflow via can be near the top surface 120 and the reagent outflow via near the bottom surface 121 or the reagent inflow via can be near the bottom surface 121 and the reagent outflow via near the top surface 120. Other permutations of via positions are possible within the scope of the invention. Altering position of the vias can provide for the air to come in from the top and fluid out the bottom, or vice versa, or for the air to “bubble” through the fluid.

It will be appreciated that the principles and methods disclosed also provide for multiple selector valve arrangements in a single cartridge. Additional selector valve arrangements can allow for central control of internal mixing of reagents with the sample material or with other reagents before entering the analyzer interface region.

The herein described passageways for moving fluids from compartment to compartment through a valve region and along the bottom surface, top surface and the body of the cartridge permits a significant number of fluid passageways to be coursed through a compact body that is easily manufactured through known techniques.

Referring now to FIG. 14, one embodiment for mounting a reading arrangement 108 within cartridge 100 will be described. FIG. 14 is an enlarged view of a portion of the cross-section view of FIG. 13. In use, reading arrangement 108, such as flow cell 109 is maintained in position within the analyzer interface region 107 by four clasps 400, 401 (visible in FIG. 6) and 402 and 403 (visible in FIGS. 13 and 14). The flow cell 109 is maintained within a gap between edges 405 and 406 of clasps 402 and 403 and sidewall 407. A similar arrangement is present on the opposite side of analyzer interface region 107, but clasps 400, 401 and sidewall 420, are not visible in FIG. 14. The distance X between edges 405 and 406 and sidewall 407 is greater than the thickness of flow cell 108. Thus, flow cell 109 can move back and forth within this space.

In this embodiment, elastomeric tubing 410 and 411, each having lumens continuous with analyzer inflow via 350 and analyzer outflow via 351, respectively, are positioned such that the elastomeric tubing 410, 411 biases flow cell 109 against the edges 405 and 406 of clasps 402 and 403 (and 400 and 401).

In use, at the analyzer interface region 107, the instrument can exert a force in the direction of arrow A to bias flow cell 109 away from clasps 400-403 and toward sidewalls 407 and 420 without causing flow cell 108 to contact sidewalls 407 and 408. In this position, flow cell 109 is not in contact with any portion of body 101 but rather is only in contact with the edge surfaces 412 and 413 of elastomeric tubing 410 and 411, respectively. Thus, flow cell 109 can “float” within analyzer interface region 107.

In some embodiments, for example in some SPR instruments, when cartridge 100 is in use, top surface 120 and bottom surface 121 are angled 15°, relative to a horizontal plane, to facilitate fluid flow into analyzer inflow via 350 and out analyzer outflow via 356.

In one embodiment, a cartridge of the invention is particularly suited for use with an analytical instrument including a surface plasmon resonance (“SPR”) detector. Examples of some analytes suitable for detection according to this embodiment include myoglobin, creatinine phosphokinase, troponin I, human chorionic gonadotropin, blood urea nitrogen, creatinine, amylase, lipase, ALT, AST, alkaline phosphatase, bilirubin, etc. In addition, blood panels including WBC, RBC, hematocrit, hemoglobin, etc. can also be performed.

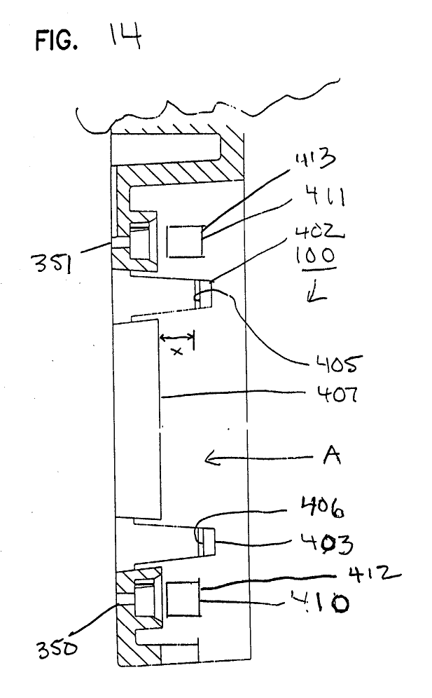

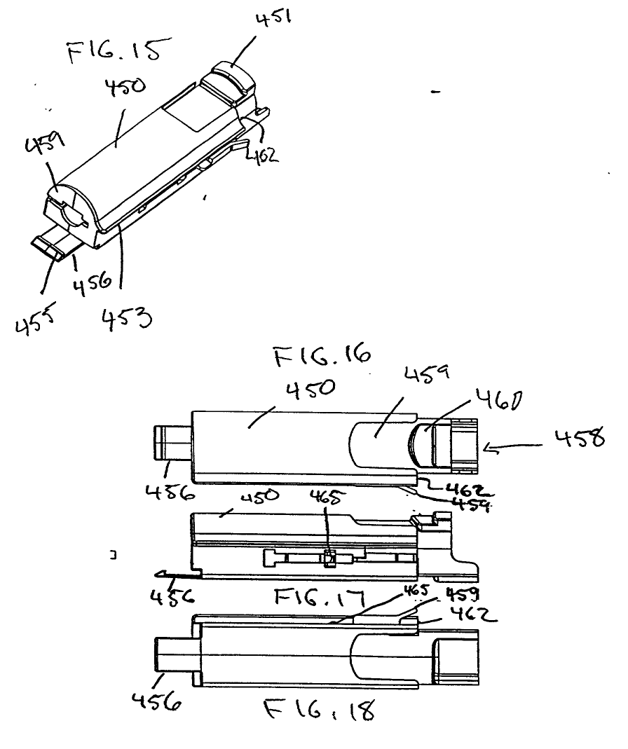

In another embodiment, the invention provides a docking arrangement 102 that can mount to a cartridge 100 as shown in FIGS. 2-5. Referring to FIGS. 15-18, FIG. 15 is a top perspective view of one embodiment of a docking arrangement 102, FIG. 16 is a top plan view, FIG. 17 a first side plan view and FIG. 18 a bottom plan view. Docking arrangement 102 includes a docking tube 450 that can slidably receive a sample housing 451. Docking tube 450 can mount to cartridge 100 by sliding lip 453 of the docking tube 450 along ridge 454 (FIG. 8) of cartridge 100 until clasps 455 of extension member 456 engage edge 457 (FIG. 7) of cartridge 100. Tab 459 can also lock against the cartridge 100 below shoulder 470 of the cartridge (FIG. 9) to prevent the docking tube from sliding off the cartridge.

Referring to FIGS. 16-18, to mount a sample collection container, such as a blood tube, to docking arrangement 102, the blood tube (not shown) is inserted into lumen 458 of sample housing 451. The top of the tube abuts against an axially directed collar that is inside lumen 458 at the leading end (not visible) of sample tube 451. As the tube is pushed into the sample housing, the leading end collar of sample tube 451 is pushed towards the leading end 459 of docking tube 450. The trailing end 462 of docking tube 450 includes a cutout 459 which acts as a track for shoulder 460 to pass as sample housing 451 is advanced within docking tube 450. When fully advanced, a rubber stopper of the blood tube (not shown) will be pierced by hollow tubes 150 and 151 (FIG. 6). After completion of an analysis, sample housing 451 can be pulled away from cartridge 100 such that the leading end collar of sample housing 451 is pulled against the stopper to disengage the stopper from hollow tubes 150 and 151. The blood tube can then be removed from sample housing 451.

If it is desired to prevent reuse of cartridge 100, after sample housing 451 is fully retracted from the leading end 459 of docking tube 450, tab 465 of sample housing 451 can engage behind the trailing end 462 of docking tube 450 to prevent re-advancement of the sample housing tube 451 within docking tube 450.

From the foregoing detailed description and examples, it will be evident that modifications and variations can be made in the products and processes of the invention without departing from the spirit or scope of the invention. Therefore, it is intended that all modifications and variations not departing from the spirit of the invention come within the scope of the claims and their equivalents.

What is claimed is:

1. A cartridge comprising:
a fluid reservoir for storage of a fluid;
a plurality of fluid passageways;
a selector valve for selectively controlling fluid flow through the fluid
passageways; and
an analyzer interface region for interfacing with a detector.

2. The cartridge according to claim 1 further comprising a sample receiving region for receiving a sample material.

3. The cartridge according to claim 1 further comprising a waste receptacle for receiving fluids passed through the cartridge.

4. The cartridge according to claim 2 wherein the sample receiving region is a reservoir integral to the cartridge.

5. The cartridge according to claim 3 wherein the waste receptacle is integral to the cartridge.

6. The cartridge according to claim 1 comprising a body including a valve chamber for receiving a valve core of the selector valve to direct fluid flow from a first fluid passageway of the plurality of fluid passageways to a second fluid passageway of the plurality of fluid flow passageways.

7. The cartridge according to claim 6 wherein the valve chamber includes a valve seat having a valve seat channel through which a fluid can flow from the first fluid passageway to the second fluid passageway.

8. The cartridge according to claim 7 wherein the valve seat channel provides for fluid flow from a driver inflow passageway to a reservoir inflow passageway.

9. The cartridge according to claim 7 wherein the valve seat channel provides for fluid flow from a reservoir outflow passageway to an analyzer passageway.

10. The cartridge according to claim 6 wherein the valve chamber includes a first valve seat channel through which a fluid can flow from the first fluid passageway to the second fluid passageway and a second valve seat channel through which a fluid can flow from a third fluid passageway to a fourth fluid passageway.

11. The cartridge according to claim 10 wherein the first fluid passageway is a driver inflow passageway, the second fluid passageway is a reagent inflow passageway, the third fluid passageway is a reagent outflow passageway and the fourth passageway is an analyzer passageway.

12. The cartridge according to claim 6 wherein the selector valve includes a fluid slot through which a fluid can flow from the first fluid passageway to the second fluid passageway.

13. The cartridge according to claim 12 wherein the valve seat channel can be in fluid communication with the fluid slot of the selector valve for fluid flow from the first fluid passageway to the second fluid passageway.

14. The cartridge according to claim 6 wherein the selector valve can be selectively positioned to direct fluid flow from the first fluid passageway to a third fluid passageway of the plurality of fluid flow passages.

15. The cartridge according to claim 1 including a conditioning region that can interface with a fluid conditioning element of an analytical instrument to condition a fluid flowing through the plurality of fluid passageways.

16. The cartridge according to claim 15 wherein the conditioning region provides for heating a fluid flowing through the plurality of fluid passageways.

17. The cartridge according to claim 16 wherein the fluid conditioned by the conditioning region is a sample material.

18. The cartridge according to claim 16 wherein the fluid conditioned by the conditioning region is a reagent fluid.

19. The cartridge according to claim 1 further comprising a filter region through which a fluid can be passed.

20. The cartridge according to claim 19 wherein the fluid passed through the filter is the sample material.

21. The cartridge according to claim 19 wherein the fluid passed through the filter is a reagent fluid.

22. The cartridge according to claim 2 wherein the sample receiving region can couple to a sample docking arrangement.

23. The cartridge according to claim 22 wherein the sample receiving region includes an arrangement for piercing a rubber stopper of a sample container in the sample docking arrangement.

24. The cartridge according to claim 1 wherein the cartridge includes a driver interface for receiving a driver to move fluid through the plurality of fluid passageways.

25. The cartridge according to claim 24 wherein the driver is air and the driver interface engages with a pump on an analytical instrument that forces air into the cartridge at the driver interface.

26. The cartridge according to claim 1 wherein the analyzer interface region includes a flow cell configured for interfacing with the detector of a surface plasmon resonance instrument.

27. The cartridge according to claim 3 wherein the waste receptacle includes a vent.

28. The cartridge according to claim 1 further comprising a body including:
a top surface and a bottom surface;
a plurality of surface channels, through body passageways and vias; and
a top membrane sealably attached to the top surface and a bottom
membrane sealably attached to the bottom surface.

29. The cartridge according to claim 27 wherein the body is injection molded.

30. The cartridge according to claim 1 wherein the detector is on an analytical instrument.

31. The cartridge according to claim 1 wherein the detector is a human.

32. A sample analysis system comprising:
an analytical instrument; and
a cartridge comprising:
a fluid reservoir for storage of a fluid;
a plurality of fluid passageways;
a selector valve for selectively controlling fluid flow through the fluid passageways;
an analyzer interface region for interfacing with a detector of the analytical instrument.

33. The sample analysis system according to claim 32 wherein the analytical instrument is a spectrophotometer.

34. The sample analysis system according to claim 32 wherein the analytical instrument is a surface plasmon resonance instrument.

35. The sample analysis system according to claim 32 wherein the cartridge comprises a body including a valve chamber for receiving a valve core of the selector valve to direct fluid flow from a first fluid passageway of the plurality of fluid passageways to a second fluid passageway of the plurality of fluid passageways.

36. The sample analysis system according to claim 35 wherein the analytical instrument includes a control center for controlling the direction of fluid flow through the selector valve.

37. The sample analysis system according to claim 32 wherein the cartridge includes a driver interface for receiving a driver to move fluid through the plurality of fluid passageways and the analytical instrument dispenses the driver to the driver interface.

38. The sample analysis system according to claim 34 wherein the driver is dispensed to the driver interface of the cartridge by a single stroke positive displacement pump.

39. A method for analyzing a sample material, the method comprising a step of:
placing a cartridge into an analytical instrument, the cartridge including:
(i) a plurality of fluid passageways
(ii) a selector valve to selectively direct flow of a fluid through the fluid passageways;
introducing a fluid into the cartridge;
driving the fluid through the cartridge with a driver;
operating the selector valve to selectively direct the flow of the fluid through the fluid passageways.

40. The method according to claim 39 wherein the fluid is a sample material.

41. The method according to claim 40 wherein the sample material is introduced into the cartridge before the cartridge is placed in the analytical instrument.

42. The method according to claim 40 wherein the sample material is introduced into the cartridge by the analytical instrument.

43. The method according to claim 39 wherein the analytical instrument includes a driver dispenser to deliver the driver to the cartridge to drive the fluid through the cartridge.

44. The method according to claim 39 wherein the analytical instrument includes a control center for operating the selector valve.

45. The method according to claim 39 wherein the analytical instrument includes a control center for controlling the driver dispenser.

46. The method according to claim 39 wherein the control center is operated by a software program.

47. The method according to claim 43 wherein the fluid is a reagent fluid.

48. The method according to claim 39 wherein the selector valve includes a valve face comprising an elastomeric material having a first side and a second side wherein at least one of the first and second sides is covered by a flexible, low-friction, high tensile strength material.
